(19) United States
(12) Patent Application Publication
(10) Pub. No.: US 2025/0288338 A1
(43) Pub. Date: Sep. 18, 2025

(54) BONE CEMENT MIXING SYSTEM WITH PUNCTURE CONNECTION AND PUMP FUNCTION

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/075,393

(22) Filed: Mar. 10, 2025

(30) Foreign Application Priority Data
Mar. 15, 2024 (EP) 24163716.4

(51) Int. Cl. A61B 17/88

(52) U.S. Cl. CPC A61B 17/8833; A61B 17/8822; A61B 2017/8838

(57) ABSTRACT

A device for producing bone cement, comprising a base part having a carrier for receiving a vessel containing monomer liquid, a bottom part having a collection region with a tip and a piston having a passage for receiving the hollow mandrel, and a cartridge for receiving a PMMA bone cement powder, wherein the cartridge comprises a septum which delimits the cartridge toward the outside; wherein the piston is configured to pump a monomer liquid from the collection region and into the cartridge through the hollow mandrel.

### CROSS-REFERENCE TO RELATED APPLICATIONS

This application claims priority pursuant to 35 U.S.C. 119(a) to European Application No. 24163716.4, filed Mar. 15, 2024, which application is incorporated herein by reference in its entirety.

### FIELD OF THE INVENTION

The present invention relates to the field of medical engineering, in particular to devices for producing a PMMA bone cement and for storing components thereof.

### PRIOR ART

Polymethyl methacrylate (PMMA) bone cements stem from the fundamental work of Charley. Polymethyl methacrylate bone cements are usually composed of a liquid monomer component and a powder component. The monomer component can contain the monomer methyl methacrylate and an activator (e.g., N,N-dimethyl-p-toluidine) dissolved therein. The powder component, also referred to as bone cement powder, can comprise one or more polymers producible on the basis of methyl methacrylate and comonomers, such as styrene, methyl acrylate or similar monomers, by polymerization, preferably suspension polymerization. The powder component may further comprise a radiopaque agent and an initiator, such as dibenzoyl peroxide. When the powder component is mixed with the monomer component, a plastically deformable dough, the actual bone cement dough, can form as a result of the polymers in the powder component swelling in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough increases until the cement dough solidifies.

Polymethyl methacrylate bone cements can be mixed in suitable mixing cups using spatulas by mixing the power component with the monomer liquid. Air bubbles may be incorporated in the bone cement dough, which can negatively influence the mechanical properties of the hardened bone cement.

In order to avoid air inclusions in the bone cement dough, a plurality of vacuum cementing systems have been described, of which the following are mentioned by way of example: U.S. Pat. Nos. 6,033,105 A, 5,624,184 A, 4,671,263 A, 4,973,168 A, 5,100,241 A, WO 99/67015 A1, EP 1 020 167 A2, U.S. Pat. No. 5,586,821 A, EP 1 016 452 A2, DE 36 40 279 A1, WO 94/26403 A1, EP 1 005 901 A2, and U.S. Pat. No. 5,344,232 A.

Cementing systems in which both the cement powder and the monomer liquid are already packed in separate compartments of the mixing systems and are not mixed together in the cementing system until immediately before the cement is applied represent a development in cementing technology. Such closed “fully prepacked mixing systems” are described in patent documents EP0380867B1, EP0796653B1, EP0692229B1, U.S. Pat. Nos. 5,997,544A, 6,709,149B1, DE69812726T2 and U.S. Pat. No. 5,588,745A, WO9416951A1, DE19718648A1, EP1741413B1, EP3054880B1, DE102009031178B3, U.S. Pat. No. 8,662,736B2, EP2269718B1, EP2281532B1 and EP3093067B1.

Fully prepacked mixing systems encounter a few challenges. It is desirable to ensure safe, reproducible transfer of the monomer liquid to the polymethyl methacrylate bone cement powder and to avoid uncontrolled mixing of the components during storage. It is advantageous if the cement powder cannot enter the line means for transferring the monomer liquid during transport and storage of the mixing system. Otherwise, the line means may become blocked when transferring the monomer as a result of the cement powder swelling and sticking together.

Blockage of the line means by swollen bone cement sticking together could interrupt the transfer of the monomer liquid and prevent the cement components from being mixed in the predetermined mixing ratio.

In the fully prepacked mixing systems according to EP0796653B1, U.S. Pat. No. 6,709,149B1, EP1741413B1, EP3054880B1, EP2269718B1 and EP2281532B1, the monomer is transferred by applying an external vacuum to the cartridge via a vacuum connection and a porous plate. This means that the monomer liquid is drawn into the cartridge by the vacuum applied. In the mixing system described in EP3093067B1, the monomer liquid can either be transferred to the cartridge by the action of a vacuum, or alternatively the monomer liquid can be transferred by being pushed by means of a manually operable pump piston integrated into the mixing system.

According to EP2281532B1, the line means for transferring the monomer liquid is prevented from being blocked by using a mesh permeable to the monomer liquid or a porous disk to block the cement powder particles, wherein the mesh or porous disk is part of a special nozzle that generates a sharp jet of monomer liquid when transferring it, which jet displaces swollen cement particles so that the monomer liquid can be completely transferred to the bone cement powder.

EP1741413B1 discloses a method for bringing a powder and a liquid component into contact, wherein a liquid container is opened by a cannula penetrating into a wall portion of the liquid container that closes a lateral hole in an inner tube element in the liquid container.

Patent document EP3636338B1 discloses a mixing system comprising a connecting cylinder configured to be selectively coupled to and removable from a mixing chamber, and wherein a valve is configured to seal the connection between the connecting cylinder and the valve when they are selectively coupled to one another, and is configured to seal the mixing chamber when the connecting cylinder is removed from the mixing chamber. EP3490700B1 discloses a similar mixing system.

### PREFERRED EMBODIMENTS

An object of the present invention is to solve one or more of the problems described above and further problems of the prior art. For example, the invention provides a device for storing and mixing bone cement, which allows for simple and reliable mixing of a monomer liquid with a powder component of a PMMA bone cement to produce a ready-to-use PMMA bone cement dough therefrom. The devices according to the invention can comprise a plurality of parts which can be easily connected to one another and separated from one another again, thereby allowing a monomer liquid to be reliably and safely transferred from one part of the device to the other part of the device. A self-sealing connection system can prevent monomer liquid from escaping or contamination from entering. The device according to the invention can offer easy handling. A monomer liquid and a powder component of a PMMA bone cement can be stored separately in the device, combined in a controlled manner and mixed together. The device can release a monomer liquid from a closed vessel in an interior of the device and transfer it to another part of the device by means of a movable piston. The device can perform these functions while securely placed on a support. The monomer liquid can be transferred in full, thereby preventing the device from being clogged with clumps of powder.

These objects are achieved by the methods, devices, kits and medical uses described herein, in particular those which are described in the claims.

Preferred embodiments of the invention will be described below.

A first embodiment relates to a device for producing bone cement, comprising a base part which comprises a container having a carrier for receiving a vessel containing monomer liquid, a bottom part having a collection region, a hollow mandrel having a tip and a piston having a passage for receiving the hollow mandrel, and a cartridge for receiving a PMMA bone cement powder, wherein the cartridge comprises a septum which delimits the cartridge toward the outside;
wherein the piston is configured to pump a monomer liquid from the collection region, through the hollow mandrel and into the cartridge.

A second embodiment relates to a device according to the first embodiment, wherein the device is designed and configured to form a fluid-conducting connection between the base part and the cartridge by piercing the septum using the hollow mandrel in order to transfer a monomer liquid from the collection region to the cartridge.

A third embodiment relates to a device according to the first or second embodiment, wherein the base part comprises a connecting channel with an outlet opening, wherein the connecting channel forms a fluid-conducting connection between the container and the collection region.

A fourth embodiment relates to a device according to the third embodiment, wherein the piston is designed and configured to close the outlet opening of the connecting channel in a liquid-tight manner when the piston is inserted into the base part.

A fifth embodiment relates to a device according to any one of the preceding embodiments, wherein the piston is designed and configured to be fully inserted into the collection region such that no dead volume remains in the collection region.

A sixth embodiment relates to a device according to any one of the preceding embodiments, wherein the base part comprises a stop to limit the movement of the piston and/or the cartridge in the base part.

A seventh embodiment relates to a device according to any one of the preceding embodiments, wherein the device comprises a filter, wherein the filter is arranged and designed to prevent particles from entering the cartridge from the base part.

An eighth embodiment relates to a device according to the seventh embodiment, wherein the septum comprises a rubber-elastic material.

A ninth embodiment relates to a device according to any one of the preceding embodiments, wherein the base part is configured to allow a monomer liquid to be transferred from the container to the collection region by means of free gravity flow.

A tenth embodiment relates to a device according to any one of the preceding embodiments, wherein the cartridge is detachably connectable to the base part.

An eleventh embodiment relates to a device according to any one of the preceding embodiments, wherein the hollow mandrel is mounted in a cylindrical holder, wherein the holder is arranged in the collection region.

A twelfth embodiment relates to a device according to any one of the preceding embodiments, wherein the collection region comprises a recess, and wherein the hollow mandrel is preferably connected in a fluid-conducting manner to the deepest point of the recess.

A thirteenth embodiment relates to a device according to any one of the preceding embodiments, wherein the device is designed and configured to transfer a monomer liquid from the base part to the cartridge while the base part is placed on a support such that the bottom part is oriented toward the support.

A fourteenth embodiment relates to a device according to any one of the preceding embodiments, wherein the container comprises an ampule breaker, wherein the ampule breaker is configured to open a glass ampule inside the container.

A fifteenth embodiment relates to a device according to any one of the preceding embodiments, wherein the ampule breaker is designed and configured to open a glass ampule inside the container by bending the container relative to the bottom part or by moving the carrier against a projection.

### BRIEF DESCRIPTION OF THE DRAWINGS

FIG. 1 shows the cross section of a device according to the invention.

FIG. 2 shows the cross section of a device according to the invention, wherein a base part comprises a projection for breaking off the head of a glass ampule.

FIG. 3 shows the cross section of a device according to the invention, wherein monomer liquid flows from a glass ampule broken open in a container and into a collection region of the base part.

FIG. 4 shows the cross section of a device according to the invention, wherein a cartridge is placed on a base part in order to pierce a septum of the cartridge by means of a hollow mandrel of the base part.

FIG. 5 shows the cross section of a device according to the invention, wherein a piston is inserted into the base part in order to close an outlet opening of a connecting channel in a liquid-tight manner.

FIG. 6 shows the cross section of a device according to the invention, wherein a piston is pushed completely into a collection region of a base part in order to substantially completely transfer a monomer liquid from the collection region to the cartridge.

FIG. 7 shows the cross section of a device according to the invention, wherein a cartridge is detached from the base part after monomer liquid has been transferred from the base part to the cartridge.

FIG. 8 shows a cross section of a cartridge comprising a mixing rod for mixing a monomer liquid with a PMMA bone cement powder.

FIG. 9 shows a cross section of a cartridge having a threaded rod and a discharge tube for dispensing bone cement dough from the cartridge.

FIG. 10 shows an embodiment of a device according to the invention which comprises a flexible container for receiving and breaking open a glass ampule containing monomer liquid.

FIG. 11 shows a device according to FIG. 10, wherein the container is bent in order to break open a glass ampule containing monomer liquid in the base part.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, all the features described herein in connection with a method are in principle also considered to be applicable to the products, devices, kits and uses described herein, and vice versa. All such considered combinations are not explicitly listed in all instances, simply in order to keep the description brief. Technical solutions known to be equivalent to the features described herein are also intended in principle to be encompassed by the scope of the invention.

A first embodiment relates to a device for producing bone cement, comprising
a base part which comprises a container having a carrier for receiving a vessel containing monomer liquid, a bottom part having a collection region, a hollow mandrel having a tip and a piston having a passage for receiving the hollow mandrel, and
a cartridge for receiving a PMMA bone cement powder, wherein the cartridge comprises a septum which delimits the cartridge toward the outside;
wherein the piston is configured to pump a monomer liquid from the collection region, through the hollow mandrel and into the cartridge.

The device according to the invention can comprise two interconnectable parts, specifically a base part and a cartridge. The base part comprises a container for receiving a monomer liquid. The container comprises a carrier for receiving a vessel containing monomer liquid, for example a glass ampule or a pouch. Such glass ampules or pouches are used as commercially available packaging for monomer liquid of a PMMA bone cement. The container can be prefilled with monomer liquid, for example packaged in a gas-tight manner in a glass ampule or a pouch. Alternatively, the container may be empty and fillable with monomer liquid by the user, for example by introducing a glass ampule containing monomer liquid into the container. The cartridge can also be empty or pre-filled with PMMA bone cement powder. The base part may comprise a transparent region. For example, the container may comprise a transparent region. The collection region can also comprise a transparent region. This may allow a user of the device to visually verify that all the monomer liquid has drained from the container into the collection region from the outside. Furthermore, the base part can comprise a fill level marking. Such a fill level marking can, for example, be arranged at the position of a desired liquid level of the collection region, which level can be achieved by transferring all of a monomer liquid from the container into the collection region.

The base part can be configured to be placed on a support. The base part comprises a bottom part, which may comprise a flat outer surface. The bottom part comprises a collection region. The collection region is preferably designed and configured to receive monomer liquid. For example, the base part can be configured to release monomer liquid from a vessel, such as a glass ampule or a foil pouch, and to transfer it to the collection region by gravity flow. The collection region may comprise a recess, for example be concavely curved, similar to a watch glass dish. The cross section of the recess can be U-shaped or V-shaped. The recess may be funnel-shaped.

The collection region preferably has a capacity for receiving the monomer liquid that exceeds the volume of the monomer liquid contained or receivable in the container. For example, the collection region may have a volume which is at least 1.1-times the volume of the monomer liquid contained or receivable in the container. For example, if the container comprises a carrier for receiving commercially available glass ampules containing 10 ml of monomer liquid, the collection region preferably has a volume of at least 11 ml. This applies, mutatis mutandis, to embodiments which comprise a carrier for receiving glass ampules containing 5 ml, 20 ml or 30 ml of monomer liquid.

The base part also comprises a hollow mandrel having a tip. The hollow mandrel preferably comprises an interior that allows a liquid to flow through the hollow mandrel.

The interior can have a diameter of 1.0 mm to 3.0 mm. The hollow mandrel can comprise outlet openings having a diameter of less than 1.5 mm, and particularly preferably less than 1.0 mm. Due to the hollow mandrel having a small diameter, a sharp jet can be formed when a monomer liquid is transferred from the collection region to the cartridge, which jet can displace smaller particles of bone cement powder, if necessary. This can prevent the hollow mandrel from becoming blocked. The hollow mandrel can comprise a beveled tip, similar to a ground cannula, with an outlet opening of the hollow mandrel preferably being located in a bevel of the tip. The hollow mandrel can comprise a round tip. One or more openings can be arranged near the tip and are connected to the interior of the hollow mandrel. Furthermore, the interior of the cavity can comprise a shoulder on which a porous pin permeable to liquids is supported. This pin can serve as a filter, as described hereinbelow. The interior of the hollow mandrel can taper toward the shoulder. The shoulder may be configured to hold the pin in the hollow mandrel. The hollow mandrel can be made of plastics or metal, e.g., stainless steel. Examples of usable plastics include Nylon 12, Nylon 6, Nylon 66 and polyethersulfone. The hollow mandrel, particularly the tip of the hollow mandrel, may be covered by a removable protective cap.

The cartridge comprises a septum which delimits the cartridge toward the outside. The septum is preferably arranged in a region of the cartridge which is designed and configured for connection to the base part. The septum can be pierced by the hollow mandrel of the base part. In a configuration of the device in which the septum is pierced by the hollow mandrel, a fluid-conducting connection can form between the base part and the cartridge. This makes it possible to transfer a monomer liquid from the collection region of the base part to the cartridge. The self-sealing function of the septum can prevent monomer liquid from escaping from the device. This also applies if the hollow mandrel is withdrawn from the septum again, as the puncture site can close automatically due to the restoring force of the septum. Furthermore, this can prevent contamination from entering the device from the outside. In one embodiment, the device is designed and configured to form a fluid-conducting connection between the base part and the cartridge by piercing the septum by means of the hollow mandrel in order to transfer a monomer liquid from the collection region to the cartridge.

The term “fluid-conducting connection” or “liquid-conducting connection” means here that a monomer liquid can flow from one part of the device to another part of the device.

The septum can comprise a rubber-elastic material. In some embodiments, the septum comprises a flexible, self-sealing polymer. The polymer may comprise a polyhalogenated olefin, a silicone, rubber, butyl rubber, ethylene propylene diene rubber (EPDM), or a similar elastomer. The septum is preferably configured to seal the cartridge in a liquid-tight manner after the septum has been pierced by the hollow mandrel. The septum is preferably stretchable and exhibits rubber-elastic properties, as is known, for example, from septa in chemical bottles. This allows the septum to heal itself after being pierced, i.e., to automatically seal.

The septum preferably comprises a biocompatible material. The septum can comprise a circular shape. In one embodiment, the septum comprises a material which can be pierced with a medical injection cannula according to ISO 7864:2016 with a force of less than 100 N. The septum is preferably connected to the cartridge in a liquid-tight manner. In one embodiment, the septum is designed and configured to close a puncture site in the septum in a liquid-tight manner after the hollow mandrel has punctured it and subsequently been removed. This can be achieved by the restoring force of a rubber-elastic material of the septum.

The cartridge may comprise a guide element, e.g., an insertion bevel, which facilitates insertion of the hollow mandrel into the septum in a desired position.

The septum can be mounted in a holder. The holder can be detachably connected to the cartridge. The septum holder can be designed as a piston. This piston may be designed and configured to dispense bone cement dough from the cartridge. The septum holder can be arranged in a cartridge head. The cartridge head can be designed as a detachable cartridge closure.

The cartridge and the base part can preferably be stored separately from one another, i.e., they are designed as separate sub-elements that can be detachably connected to one another. This can prevent fatigue of the septum's restoring force during storage and facilitate the automatic closure of the septum. Unwanted leakage of monomer liquid from the cartridge after the hollow mandrel has been removed from the septum can be prevented. Furthermore, separate storage has the advantage that packaging materials with small dimensions can be used.

The device also comprises a piston. The piston can be arranged inside the base part. The piston may comprise a passage for receiving the hollow mandrel. The passage can extend from an interior of the base part toward the outside. In a basic configuration of the device, the piston may be configured to receive the tip of the hollow mandrel in the passage. In such a basic configuration of the device, no external forces are exerted on the device. In this state, the tip of the hollow mandrel is preferably arranged inside the piston so that a user is protected from injury as a result of contact with the tip. In one embodiment, the piston is configured to release the tip of the hollow mandrel from the passage when the cartridge is pushed onto the piston. This allows the cartridge to apply pressure to the piston to release the hollow mandrel and pierce the septum of the cartridge. In this case, the tip of the hollow mandrel can protrude from the passage. This allows the cartridge and base part to be connected to one another in a fluid-conducting manner with no risk of injury in order to pass a monomer liquid into the cartridge. The piston is preferably configured to be inserted through the cartridge and into the base part when the cartridge is connected to the base part. The piston can accordingly be moved by the cartridge exerting pressure on the piston.

In some embodiments, the tip of the hollow mandrel is protected by a detachable cover.

According to the invention, the piston is configured to pump a monomer liquid from the collection region, through the hollow mandrel and into the cartridge. The piston may be configured to increase the pressure inside the collection region such that the pressure inside the collection region exceeds the pressure in the cartridge.

In some embodiments, the piston is movably arranged in the base part. As a result of pressure on the piston, the piston can be movable preferably in the direction of the collection region of the base part. In this case, the piston can be moved in the direction of the interior of the base part or in the direction of a supporting surface of the base part. This movement can increase the pressure in the collection region in order to pump a monomer liquid from the collection region, through the hollow mandrel and into the cartridge.

In some embodiments, the piston is designed and configured to be fully inserted into the collection region, so that no dead volume remains in the collection region. For this purpose, a side of the piston which is oriented toward the collection region can have a surface shape which complements a surface shape of the collection region. For example, an aperture can be arranged in the piston which is complementary to a holder of the hollow mandrel. Furthermore, an outer wall of the piston can be arranged to complement an inner wall of the collection region. The piston may comprise a raised portion that complements a recess in the collection region. This allows for essentially complete transfer of monomer liquid from the collection region to the cartridge, since only a small dead volume remains inside the base part (in particular inside the hollow mandrel) when the monomer liquid is pumped from the base part into the cartridge by the movement of the piston.

In one embodiment, the base part comprises a spring which is arranged and configured for movably mounting the piston within the base part. This allows the piston to be held in a position in which the tip of the hollow mandrel is received in the passage of the piston, as described above, and is thus held inside the piston in a way that prevents injury. The spring can be a spiral spring. In one embodiment, the spring surrounds the hollow mandrel in a portion of the hollow mandrel that is located outside the passage of the piston. The spring can be arranged between the bottom part and the piston.

In an embodiment in which the hollow mandrel is mounted in a cylindrical holder, as described herein, the spring can be arranged between this holder and the piston. The spring can make it easier to separate the base part and the cartridge. Furthermore, the tip of the hollow mandrel can be automatically moved back into the cavity by the spring as soon as no more external forces are acting on the piston, e.g., when the user no longer actively pushes the cartridge against the base part. The spring can also be configured to hold the piston in a position in which the connecting channel is open, i.e., wherein the connecting channel forms a fluid-conducting connection between the container and the collection region, and the piston does not tightly close the outlet opening of the connecting channel.

In one embodiment, the base part comprises a stop. The stop may be designed and configured to limit the movement of the piston and/or the cartridge in the base part. The stop can be configured to hold the piston in the base part. In an embodiment in which the base part comprises a spring, the stop can limit the travel of the piston in the direction of the force applied by the spring. This can prevent the piston from being pushed out of the base part by the spring. In a basic configuration of the device, in one embodiment the stop is configured to hold the piston in a position in which the piston is flush with an outer surface of the base part. In one embodiment, the stop is configured to hold the piston in a position in which the tip of the hollow mandrel is received in the passage of the piston. Furthermore, the stop can be designed and configured to limit the travel of the cartridge into the interior of the base part. This allows the cartridge to be placed on the base part in a desired position without the cartridge and base part moving with respect to one another. The stop can also be designed and configured to transfer a force from the cartridge to the piston. This allows the piston to be pushed into the base part by means of the cartridge. The cartridge may comprise a projection designed and configured to engage in the stop.

A detachable connecting element may be provided to hold the cartridge on the base part in a desired position. In such a desired position, the cartridge can be flush with the stop. This connecting element can prevent the cartridge from being pushed out of the base part by the spring, if necessary. Thus, in such an embodiment, the user does not have to compensate for the pushing force exerted by the spring with their own strength when the device is being used in order to hold the cartridge in position.

In one embodiment, the base part comprises a connecting channel having an outlet opening. The connecting channel can form a fluid-conducting connection between the container and the collection region. Thus, a monomer liquid can be transferred from the container to the collection region via the connecting channel. The connecting channel preferably comprises an outlet opening, wherein the outlet opening is preferably arranged at an end of the connecting channel which points in the direction of the collection region. Preferably, the connecting channel is designed and configured to transfer monomer liquid from the container to the collection region by means of free gravity flow. This applies in particular to an arrangement of the device in which the base part is placed on a support so that the bottom part is oriented toward the support, as described herein. “Free gravity flow” means in this case that a monomer liquid can pass from the container into the collection region by the action of gravity alone, without the need for, for example, a pressure difference between the container and the collection region. Preferably, the devices described herein allow all of the monomer liquid to be transferred from the container to the collection region by free gravity flow within 20 seconds. The monomer liquid can then be transferred from the collection region into the cartridge by being pumped by the piston, as described herein.

In one embodiment, the piston is designed and configured to close the outlet opening of the connecting channel in a liquid-tight manner when the piston is inserted into the base part. By moving the piston toward the collection region, a lateral surface of the piston can be brought into contact with the outlet opening of the connecting channel in order to close the outlet opening. In such an arrangement of the device there is no fluid-conducting connection between the container and the collection region. In this configuration, the collection region is sealed with respect to the container by the piston; a fluid-conducting connection only exists between the collection region and the hollow mandrel. This allows the piston to pump monomer liquid from the collection region through the hollow mandrel, since in this arrangement pressure equalization with the container cannot occur via the connecting channel. Accordingly, the device according to the invention can be designed and configured to dispense monomer liquid from the container into the collection region in a first arrangement of the piston and to pump monomer liquid from the collection region through the hollow mandrel in a second arrangement of the piston. The hollow mandrel can be used to pierce the septum of the cartridge in order to transfer monomer liquid from the base part to the cartridge.

In one embodiment, the device comprises a filter. The filter can be arranged in the base part. For example, the filter can be arranged between the container and the hollow mandrel. The filter is preferably designed and configured to prevent particles from the base part from entering the cartridge. For example, the filter may be arranged and configured to retain glass fragments from a glass ampule broken open in the base part. The filter can be, for example, a metal wire netting or a mesh made of a plastics fabric. The filter can also be a disk made of metal or plastics and provided with expediently dimensioned holes. Furthermore, open-pore foams, such as Porex, can be used. Preferably, the filter should be made of a material which is not attacked by the monomer liquid located in a receivable glass ampule.

In one embodiment, the container comprises an ampule breaker. The ampule breaker may be configured to open a glass ampule inside the container. The container may also comprise another means for releasing a monomer liquid from a vessel, e.g., from a foil pouch. This may, for example, be a mandrel suitable for piercing a foil pouch, as described herein in more detail elsewhere.

In one embodiment, the ampule breaker is designed and configured to open a glass ampule inside the container by bending the container relative to the bottom part. For this purpose, the container can comprise a flexible housing part which allows a force to be exerted on a glass ampule in the container. The ampule breaker may comprise a rigid housing part or projection against which a glass ampule may be pushed to break open the glass ampule.

In one embodiment, the container is configured to allow a monomer liquid to be transferred from the container to the collection region by free gravity flow.

In one embodiment, the connecting channel is arranged at an angle of 20° to 80°, preferably 21° to 55° to the bottom part. This angle is defined by the inner side of each container and bottom part where the connecting channel and bottom part merge into one another in the direction of the bottom part (i.e., in the direction of the “bottom” of the base part) at a vertical side wall of the bottom part. This corresponds to the point where the monomer liquid flows from the connecting channel into the collection region when the device is used as intended. The container is preferably arranged opposite the bottom part, i.e., oriented “upward” when the device is used as intended, in order to allow the monomer liquid to flow into the collection region. For example, the connecting channel can be arranged relative to the bottom part in a similar way to a wing of a wing nut. The base part can, for example, comprise two or more containers and connecting channels. In one embodiment, the base part comprises two connecting channels, each of which is arranged in a Y shape with respect to the bottom part. The piston can be arranged between the containers, i.e., between the two connecting channels which form the “Y-shaped arms.”

The container may be arranged so as to be located above the collection region in a configuration of the base part in which the base part is placed on a support with the bottom part oriented toward the support. In particular, the outlet opening of the connecting channel can also be arranged above the collection region in such a configuration. This allows monomer liquid to reliably drain from the container into the collection region. Furthermore, this allows an arrangement of the piston in which the outlet opening of the connecting channel is sealed by the piston, and monomer liquid can be received in the collection region, i.e., a sufficient amount of free volume is available in the collection region to receive monomer liquid.

In one embodiment, a longitudinal axis of the connecting channel is arranged at an angle of 20° to 80°, preferably 21° to 55°, to an axis that runs perpendicularly to a supporting surface of the bottom part. At an angle of 21° to 55°, the formation of a meniscus by the monomer liquid can in particular be prevented.

In one embodiment, the cartridge can be detachably connected to the base part. For this purpose, appropriate connecting elements, such as a thread, can be provided on the cartridge and the base part. The connecting elements can be arranged in such a way that they allow the hollow mandrel to be connected to the septum. Furthermore, the device can comprise a guide element which facilitates the connection of the base part to the cartridge in a desired orientation. Preferably, the base part and the cartridge can be connected to one another in such a way that the piston and the septum are flush with one another. For this purpose, for example, the base part can comprise a protruding region which is designed and configured to engage in the cartridge.

In one embodiment, the base part comprises a line element, wherein the line element is configured to establish a fluid-conducting connection between the collection region and the hollow mandrel. The line element allows monomer liquid to be transferred from the collection region to the hollow mandrel, preferably by free gravity flow. The line element can be designed, for example, as a hose, tube or passage. In an embodiment in which the hollow mandrel is mounted in a cylindrical holder, as described herein, the line element can be designed as a passage in the holder.

In one embodiment, the device is designed and configured to transfer a monomer liquid from the base part to the cartridge while the base part is placed on a support such that the bottom part is oriented toward the support. The containers and the cartridge preferably point “upward,” i.e., in the opposite direction to the support and against the direction of gravity. The monomer liquid can therefore flow from the container and into the collection region by the effect of gravity.

In some embodiments, the cartridge comprises a PMMA bone cement powder. The PMMA bone cement powder is preferably arranged in an interior of the cartridge.

In some embodiments, the container comprises a monomer liquid. The monomer liquid is preferably arranged in an interior of the container. The monomer liquid may be arranged in a glass ampule or in a sealed foil pouch inside the container.

In some embodiments, the container further comprises a filter permeable for a monomer liquid. The filter is preferably arranged and configured to prevent glass splinters from the container from entering the cartridge. The filter can, for example, be arranged between the carrier and the connecting channel.

The device can be configured to receive a glass ampule containing monomer liquid. The device can further be configured to break open a glass ampule containing monomer liquid. It is possible to receive and break open the glass ampule, in particular inside the container. For this purpose, the container can comprise a receptacle for a glass ampule. The container may comprise a means for breaking open a glass ampule. For example, the container may comprise a projection arranged and configured to break off the head of a glass ampule. The container may comprise a catch region for broken glass. The filter can be arranged between this catch region and the connecting channel.

The filter may be configured to retain fragments of a glass ampule in the container. This can prevent glass splinters from entering the cartridge. This can prevent contamination of bone cement dough with glass splinters.

The monomer liquid filters described herein may be arranged either in the container or at another position on the base part, or both. For example, the container may comprise a first filter, and the line element connecting the collection region to the hollow mandrel may comprise a second filter. The second filter may have a smaller void volume than the first filter.

In some embodiments, the device comprises a mixing rod. The mixing rod is preferably configured for mixing monomer liquid and PMMA bone cement powder. The mixing rod may comprise blades. The blades can facilitate homogeneous mixing of the monomer liquid and PMMA bone cement powder. The blades are preferably arranged at an end of the mixing rod, which is arranged inside the cartridge. The mixing rod can comprise one, two or more than two blades. The mixing rod may comprise a handle at an end of the mixing rod which is preferably arranged outside the cartridge. The mixing rod is preferably movably arranged in the cartridge.

The mixing rod can comprise a discharge tube. Here, the discharge tube can be designed and configured to dispense a bone cement dough mixed in the device (i.e., a bone cement dough mixed from monomer liquid and PMMA bone cement powder). Preferably, the discharge tube is closable. This can prevent bone cement dough from undesirably escaping from the cartridge during mixing. The discharge tube can be designed as a cavity inside the mixing rod. In some embodiments, the discharge tube can be closed by a removable core. Accordingly, the mixing rod can comprise a detachable, rod-shaped core, for example a metal core. The core can also improve the strength of the mixing rod.

The mixing rod may comprise a predetermined breaking point to allow the mixing rod to be broken off after the mixing process. The predetermined breaking point is preferably located outside the cartridge so that the mixing rod can be broken off from the outside without opening the cartridge.

In some embodiments, the container further comprises a carrier for receiving an ampule. Such ampules can in particular be glass ampules in which monomer liquid is commercially available. The carrier is preferably arranged and configured to allow an ampule to be broken open and monomer liquid to drain from the ampule and through the outlet and the discharge tube by free gravity flow.

In some embodiments, the container further comprises a means for opening a foil pouch or glass ampule inside the device. An example of a means for opening a foil pouch is a mandrel which is suitable for piercing a foil pouch. Such a mandrel can be made, for example, from a rigid plastics material or from metal. An example of a means for opening a glass ampule is a projection against which the head of a glass ampule can be pushed to break it open. A means for opening a glass ampule may comprise a flexible housing part such that the glass ampule can be broken from the outside by hand by deforming the flexible housing part. A means for opening a glass ampule can also comprise a movably arranged element, such as a piston, which can push the glass ampule against a hard housing part, e.g., a projection, and thus break it open. Means for opening a foil pouch or a glass ampule are further disclosed in DE19532015A1, WO9718031A1, EP2404864B1 and WO2010012114A1.

In one embodiment, the container comprises a projection arranged in the container which is designed and configured to break open a glass ampule that can be received in the container, and further comprises a carrier that is movably arranged in the container and intended for receiving a glass ampule, wherein the carrier is designed and configured to break open a glass ampule that can be received in the holder by pushing the glass ampule against the projection. The carrier can, for example, be designed as a movable piston.

In one embodiment, the container comprises a flexible region and a rigid region, wherein the flexible region is configured to move a glass ampule received in the container against the rigid region to break open the glass ampule.

Furthermore, the base part can comprise a ventilation opening. The ventilation opening can be arranged in the container. Preferably, the ventilation opening is arranged in the base part in such a way that contact between the ventilation opening and the monomer liquid is avoided. The ventilation opening can be designed either as a simple opening or as a valve. The opening can be covered on the inside by a gas-permeable porous material so as to prevent particles from entering the container from the outside, and at the same time allowing air to be exchanged through the porous material is possible. The valve can, for example, be designed to allow air to pass only in the direction of the interior of the container. This can improve the process of transferring the monomer liquid into the collection region. Furthermore, a unidirectional valve can prevent unwanted leakage of monomer liquid from the device.

A further aspect relates to a method for producing a bone cement dough, wherein the method comprises the following steps:
providing a device according to any one of the embodiments described herein; Optionally introducing a vessel containing a monomer liquid into the container; optionally introducing a PMMA bone cement powder into the cartridge; bringing the cartridge into contact with the base part;
puncturing the septum with the hollow mandrel so that a fluid-conducting connection is formed between the collection region and the cartridge;
optionally arranging the device so that the monomer liquid is transferred from the container to the collection region by free gravity flow;
pressing the cartridge against the piston so that monomer liquid is pumped from the container into the cartridge;
mixing the monomer liquid with the PMMA bone cement powder in the cartridge to obtain a bone cement dough.

A further aspect relates to a method for producing a bone cement dough, wherein the method comprises the following steps:
providing a device comprising a base part with a vessel containing monomer liquid, a movable piston having a passage and a hollow mandrel, and a cartridge having a septum and a PMMA bone cement powder; inserting the hollow mandrel through the septum so that a fluid-conducting connection is formed between the collection region and the cartridge;
pressing the cartridge against the piston so that monomer liquid is pumped from the container into the cartridge;
mixing the monomer liquid with the PMMA bone cement powder in the cartridge to obtain a bone cement dough.

A further aspect relates to the use of a device as described herein or to the method described above for producing bone cement dough, in particular PMMA bone cement dough. Here, a monomer liquid can be mixed with a PMMA bone cement powder within the device to form a bone cement dough. Such a bone cement dough can be dispensed from the device and hardened to form PMMA bone cement at a target location.

### EXAMPLES

The invention is further illustrated below using examples which are, however, not to be understood as limiting. It will be apparent to a person skilled in the art that other equivalent means may be used similarly in place of the features described here.

### FIGURES

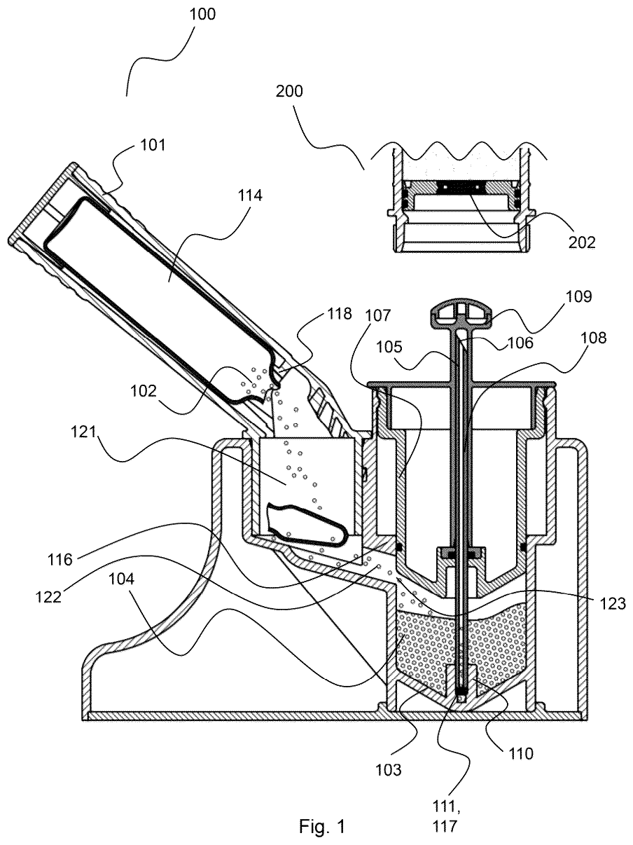

FIG. 1 shows the cross section of a device according to the invention. The device comprises a base part 100 having a container 101 for receiving a monomer liquid. A carrier 118 is arranged in the container 101 and holds a glass ampule 114 containing monomer liquid 102. The container 101 is bendable so that it can break off the head of a glass ampule received in the carrier and thereby release a monomer liquid from the glass ampule.

The base part also comprises a bottom part 103 which comprises a flat supporting surface for placing the base part on a support. A collection region 104 for receiving a monomer liquid is arranged on the bottom part. The collection region comprises a recess, with a hollow mandrel 105 that is held in a cylindrical holder 110 being arranged at the deepest point of this recess. The hollow mandrel is connected to the collection region in a liquid-conducting manner, so that monomer liquid can flow from the collection region into the hollow mandrel. The container also comprises a catch region 121 for receiving glass fragments such as a broken ampule head 115 to absorb and retain them therein. The container is connected via a connecting channel 122 to the collection region 104 in a liquid-conducting manner. Thus, a monomer liquid 102 can flow from the container 101 into the collection region 104. If the device is placed on a support by means of the bottom part 103, monomer liquid can flow from the container and into the collection region by means of gravity flow. Therefore, a pressure difference is not required to transfer the monomer liquid from the container to the collection region. The connecting channel 122 comprises an outlet opening 123 at an end of the connecting channel which extends toward the collection region 104. The outlet opening 123 is arranged above the collection region 104. The base part 100 also comprises a piston 107 having a passage 108. The hollow mandrel 105 can be received in the passage 108. The hollow mandrel 105 can produce a liquid-conducting connection between the collection region 104 and a cartridge 200. In the arrangement shown here, the hollow mandrel 105 passes through the passage 108. The hollow mandrel 105 comprises a tip 106. The tip 106 is covered by a removable cover 109 to prevent injury to the user.

The device also comprises a cartridge 200 which comprises a septum 202 that delimits the cartridge toward the outside.

The cartridge 200 can be detachably connected to the base part 100 such that the tip 106 of the hollow mandrel 105 pierces through the septum 202 and thereby creates a liquid-conducting connection between the collection region 104 and an interior of the cartridge 200. The piston 107 is configured to pump a monomer liquid from the collection region, through the hollow mandrel 105 and into the cartridge 200. The piston 107 is movably arranged in the base part 100. The piston 107 can be inserted into the base part 100 in the direction of the collection region 104 or in the direction of the bottom part 103, thereby exerting pressure on the collection region 104 such that a monomer liquid can flow through the hollow mandrel 105 from the collection region 104 into the cartridge 200.

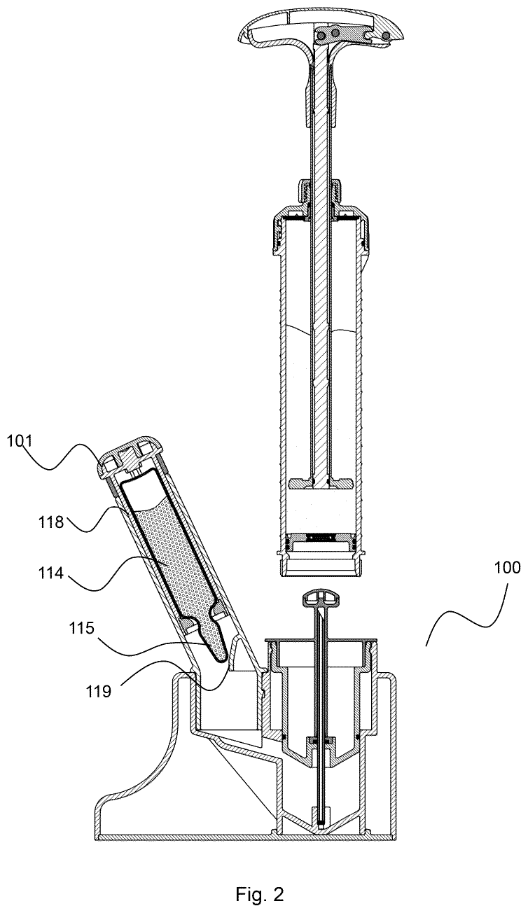

FIG. 2 shows the cross section of a device according to the invention, wherein a base part 100 comprises a projection 119 for breaking off the head 115 of a glass ampule 114. The container 101 comprises a carrier 118 which is movably arranged in the container 101. The carrier is arranged and configured to push a glass ampule 114 against the projection 119 in order to break off the head 115 of the glass ampule 114. A removable clip holder can prevent unintentional movement of the carrier when the device is not in use.

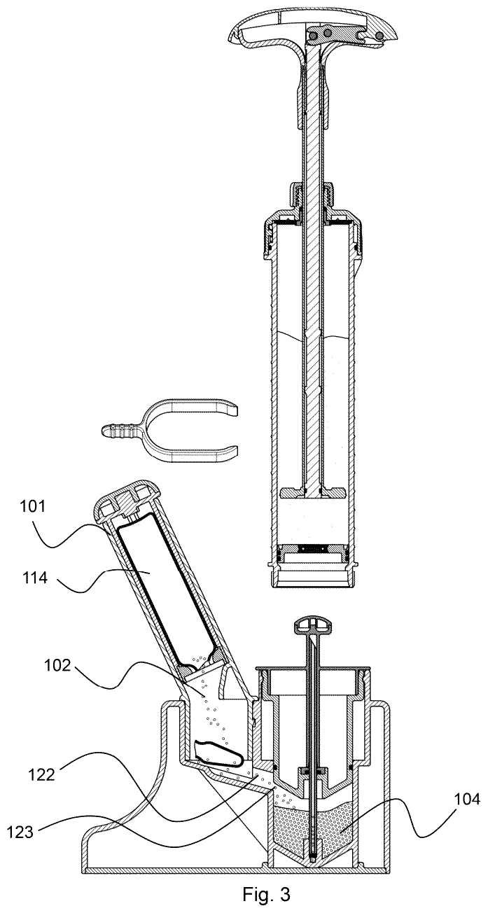

FIG. 3 shows the cross section of a device according to the invention, wherein monomer liquid 102 flows from a glass ampule 114 broken open in a container 101 into a collection region 104 of the base part 100 after the glass ampule has been broken open, as shown in FIG. 2 and described above. Monomer liquid 102 flows out of the glass ampule 114 via a connecting channel 122, through an outlet opening 123 of the connecting channel 122 and into the collection region 114.

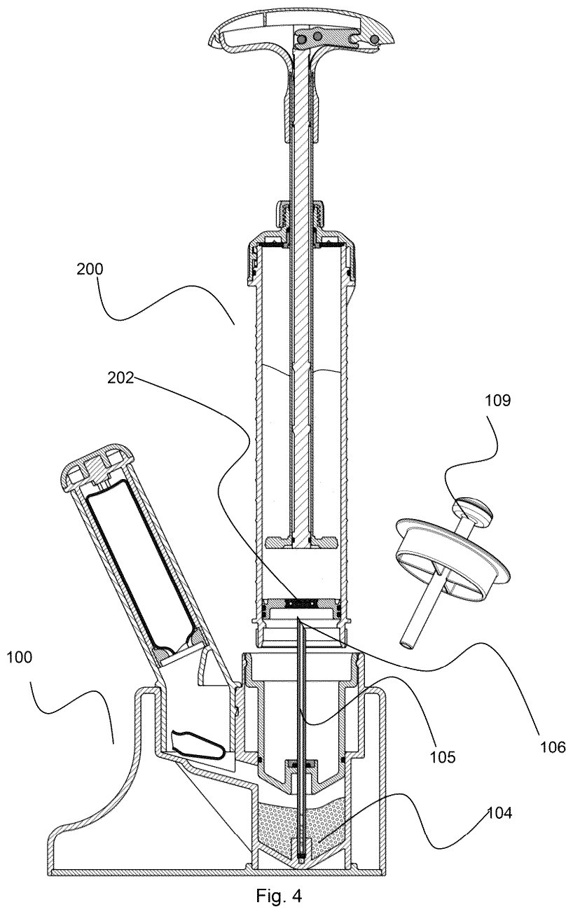

FIG. 4 shows the cross section of a device according to the invention, wherein a cartridge 200 is placed on a base part 100 to pierce a septum 202 of the cartridge 200 by means of a hollow mandrel 105 of the base part. The cover 109 is removed from the hollow mandrel 105 to release the tip 106 of the hollow mandrel 105.

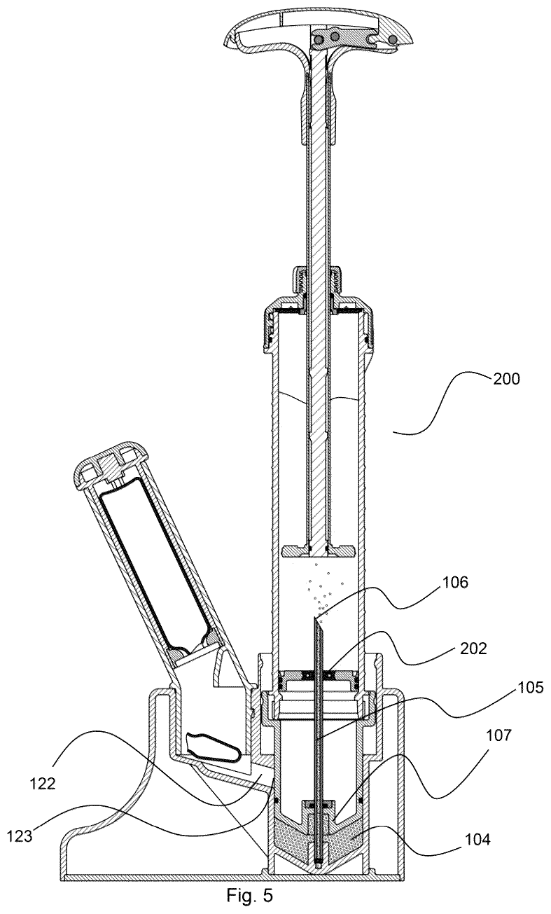

FIG. 5 shows the cross section of a device according to the invention, wherein a piston 107 is inserted into the base part 100 to close an outlet opening 123 of a connecting channel 122 in a liquid-tight manner. One side of the piston ends with the outlet opening 123 to close the outlet opening. The tip 106 of the hollow mandrel pierces through the septum 202 such that a liquid-conducting connection is established between the collection region 104 and the interior of the cartridge 200. The piston 107 exerts a pushing force on the collection region 104 to pump monomer liquid 102 from the collection region 104 and into the cartridge 200 through the hollow mandrel 105.

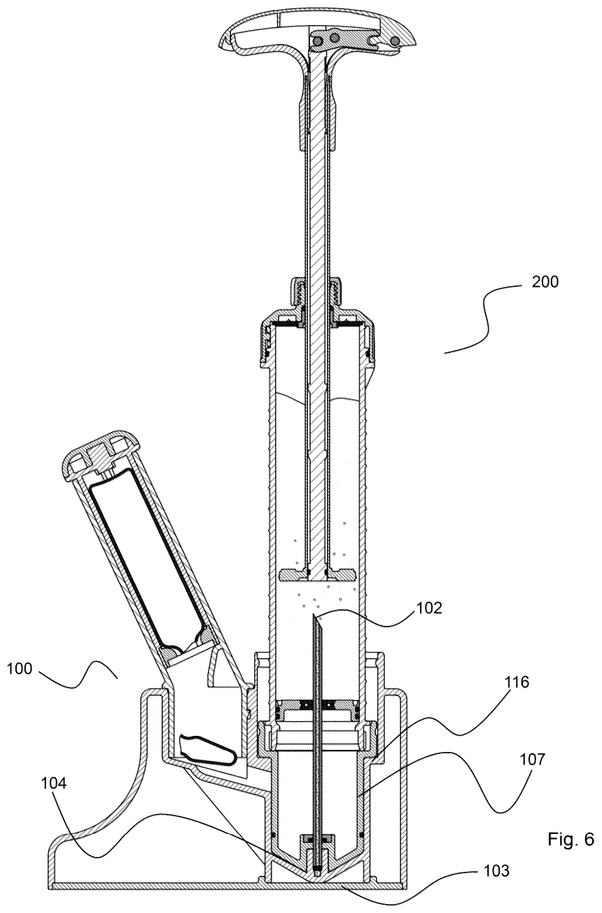

FIG. 6 shows the cross section of a device according to the invention, wherein a piston 107 is fully inserted into a collection region 104 of a base part 100 in order to substantially completely transfer a monomer liquid 102 from the collection region 104 to the cartridge 200. The front of the piston 107 substantially completely ends with a surface of the collection region 104. A projection of the piston 107 engages in a complementary stop 116 of the base part 100, thereby limiting the travel of the piston 107 toward the collection region 104 or the bottom part 103. This can prevent damage to the device.

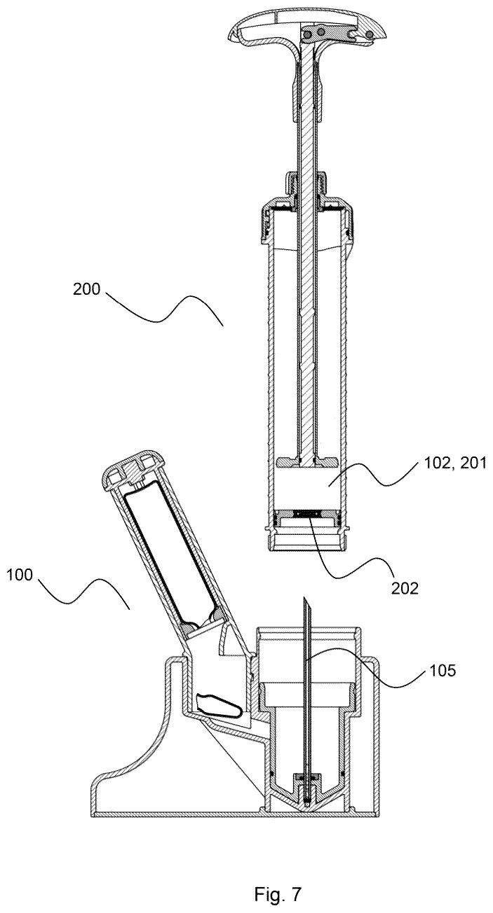

FIG. 7 shows the cross section of a device according to the invention, wherein a cartridge 200 is detached from the base part 100 after all the monomer liquid 102 has been transferred from the base part 100 to the cartridge 200. Separating the cartridge 200 and the base part 100 can facilitate the process of mixing the monomer liquid and PMMA bone cement powder 201 in the cartridge. The septum 202 comprises a rubber-elastic material such that the septum can automatically close again after the hollow mandrel 105 has been withdrawn and seals the cartridge 200 toward the outside.

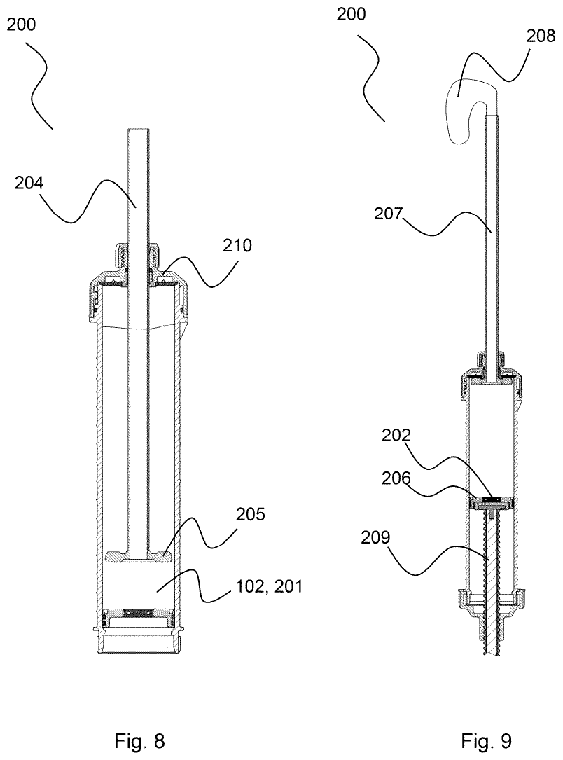

FIG. 8 shows a cross section of a cartridge 200 comprising a mixing rod 204 for mixing a monomer liquid 102 with a PMMA bone cement powder 201 in an interior of the cartridge 200.

The mixing rod 204 passes through a cartridge head 210. The mixing rod 204 is movably arranged in the cartridge 200 and comprises a mixing disk 205 which is arranged inside the cartridge 200 at one end of the mixing rod 204 to improve the mixing process of the monomer liquid and PMMA bone cement powder in the cartridge 200. By mixing monomer liquid and PMMA bone cement powder, a bone cement dough can be prepared in the cartridge.

FIG. 9 shows a cross section of a cartridge 200 having a threaded rod 209 and a discharge tube 207 for dispensing bone cement dough 208 from the cartridge 200. The threaded rod 209 is configured to move a holder 206, which supports the septum 202, within the cartridge in order to dispense bone cement dough 208 to the outside from the cartridge 200 via the discharge tube 207.

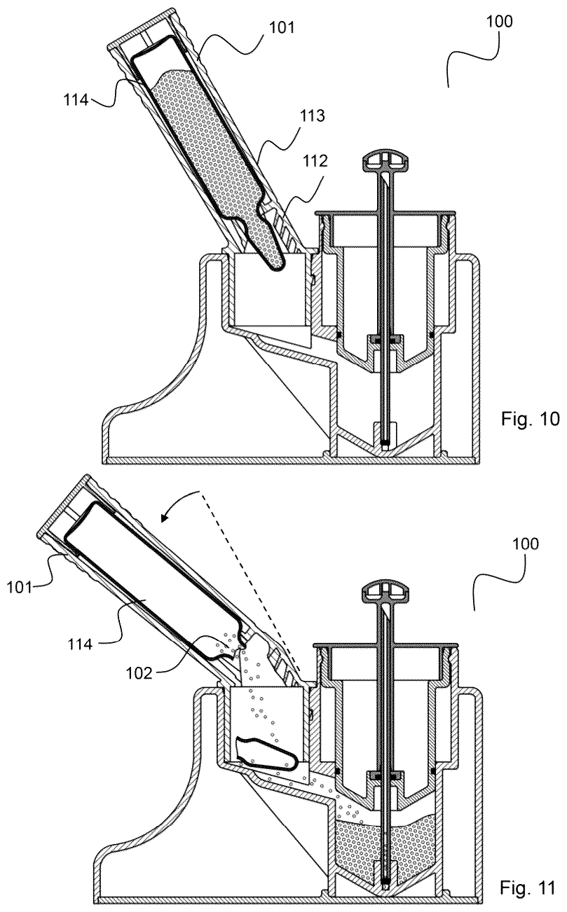

FIG. 10 shows an embodiment of a device according to the invention, which device comprises a flexible container 101 for receiving and breaking open a glass ampule 114 containing monomer liquid 102. The container 101 comprises a flexible part 112 and a rigid part 113.

FIG. 11 shows a device according to FIG. 10, wherein the container 101 is bent to form a glass ampule 114 containing monomer liquid 102 in the base part 100. The bending movement of the container 101 is shown by an arrow.

### LIST OF REFERENCE NUMERALS

100 base part
101 container
102 monomer liquid
103 bottom part
104 collection region
105 hollow mandrel
106 tip
107 piston
108 passage
109 cover
110 hollow mandrel holder
111 line element
112 flexible region of the container
113 rigid region of the container
114 vessel
115 ampule head
116 stop
117 filter
118 carrier
119 projection
120 contact region
121 catch region
122 connecting channel
123 outlet opening
200 cartridge
201 PMMA bone cement powder
202 septum
203 connection for vacuum source
204 mixing rod
205 mixing disc
206 septum holder
207 discharge tube
208 bone cement dough
209 threaded rod
210 cartridge head

1. A device for producing bone cement, comprising
a base part which comprises a container having a carrier for receiving a vessel containing monomer liquid, a bottom part having a collection region, a hollow mandrel having a tip and a piston having a passage for receiving the hollow mandrel, and
a cartridge for receiving a PMMA bone cement powder, wherein the cartridge comprises a septum which delimits the cartridge toward the outside;
wherein the piston is configured to pump a monomer liquid from the collection region, through the hollow mandrel and into the cartridge.

2. The device according to claim 1, wherein the device is designed and configured to form a fluid-conducting connection between the base part and the cartridge by piercing the septum by means of the hollow mandrel in order to transfer a monomer liquid from the collection region to the cartridge.

3. The device according to claim 1, wherein the base part comprises a connecting channel having an outlet opening, wherein the connecting channel forms a fluid-conducting connection between the container and the collection region.

4. The device according to claim 3, wherein the piston is designed and configured to close the outlet opening of the connecting channel in a liquid-tight manner when the piston is inserted into the base part.

5. The device according to claim 1, wherein the piston is designed and configured to be fully inserted into the collection region so that no dead volume remains in the collection region.

6. The device according to claim 1, wherein the base part comprises a stop for limiting the movement of the piston and/or the cartridge in the base part.

7. The device according to claim 1, wherein the device comprises a filter, wherein the filter is arranged and designed to prevent particles from the base part entering the cartridge.

8. The device according to claim 1, wherein the septum comprises a rubber-elastic material.

9. The device according to claim 1, wherein the base part is configured to allow a monomer liquid to be transferred from the container to the collection region by means of free gravity flow.

10. The device according to claim 1, wherein the cartridge is detachably connectable to the base part.

11. The device according to claim 1, wherein the hollow mandrel is mounted in a cylindrical holder, wherein the holder is arranged in the collection region.

12. The device according to claim 1, wherein the collection region comprises a recess, and wherein the hollow mandrel is preferably connected in a fluid-conducting manner to the deepest point of the recess.

13. The device according to claim 1, wherein the device is designed and configured to transfer a monomer liquid from the base part to the cartridge while the base part is placed on a support such that the bottom part is oriented toward the support.

14. The device according to claim 1, wherein the container comprises an ampule breaker, wherein the ampule breaker is configured to open a glass ampule inside the container.

15. The device according to claim 14, wherein the ampule breaker is designed and configured to open a glass ampule inside the container by bending the container relative to the bottom part or by moving the carrier against a projection.
